(19) United States
(12) Patent Application Publication
(10) Pub. No.: US 2017/0226304 A1
(43) Pub. Date: Aug. 10, 2017

(54) METHOD FOR PREPARING FOAM FOR SUPPORTING LIQUID-PHASE COSMETIC MATERIAL AND SAME FOAM

(71) Applicants: COSMAX CO., LTD., Gyeonggi-do (KR); COREAPUFF MANUFACTURING CO., LTD., Gyeonggi-do (KR)

(72) Inventor: Jong Chill LEE, Gyeonggi-do (KR)

(73) Assignees: COSMAX CO., LTD., Gyeonggi-do (KR); COREAPUFF MANUFACTURING CO., LTD., Gyeonggi-do (KR)

(21) Appl. No.: 15/502,669

(22) PCT Filed: Oct. 22, 2015
(86) PCT No.: PCT/KR2015/011202

§ 371 Date: Feb. 8, 2017

(30) Foreign Application Priority Data
Feb. 16, 2015 (KR) 10-2015-0023504
Oct. 12, 2015 (KR) 10-2015-0142023

(51) Int. Cl. C08J 9/04; A61Q 19/00; A61K 8/04

(52) U.S. Cl. CPC C08J 9/04; A61K 8/046; A61Q 19/00; A61K 2800/805; C08J 2371/00; C08J 2467/00

(57) ABSTRACT

The present invention relates to a method for preparing a foam for impregnating a liquid-phase cosmetic material, wherein the foam can be used while impregnating a liquid-phase or solid-phase cosmetic composition, such as a solution, an emulsion, a gel, a cream, or a suspension. According to the present invention, disclosed is a method for preparing a foam for impregnating a liquid-phase cosmetic material, the method comprising the steps of: (a) preparing a polyester polyol obtained by putting a polyester polyol in a chamber, followed by deflation at a temperature of 55-65° C. and mitigation under conditions of a pressure of 0.1-0.2 kPa for 60 minutes, and then lowering the temperature to 25-35° C. followed by supply of nitrogen and mitigation under conditions of a pressure of 2-3 kPa for 48 hours; (b) preparing a polyether polyol obtained by putting a polyether polyol in a chamber, followed by mitigation under conditions of a temperature of 15-25° C. and a pressure of 0.1-0.2 kPa for 60 minutes, supply of nitrogen, and mitigation under conditions of ,a pressure of 2-3 kPa for 48 hours; (c) injecting and preparing a foaming agent, a catalyst, and a surfactant in tanks according to capacity demands, respectively; (d) sequentially putting, in a mixing and stirring tank, the polyester polyol, polyether polyol, foaming agent, catalyst, and surfactant at a mixing ratio, and then performing continuous foaming with stirring at 5000 rpm under conditions of a temperature of 22-24° C. and a tank internal pressure of 3 kPa; (e) aging, for 48 hours, a foam formed after the completion of the foaming step; and (f) finishing the foam to manufacture a product.

### TECHNICAL FIELD

The present invention relates to a method of preparing a foam for impregnating a liquid-phase cosmetic material, which enables the foam to have excellent resistance to oil and water when the foam is used to impregnate a liquid-phase cosmetic composition such as a solution, an emulsion, a gel, a cream, a suspension, etc., and therefore, allows the foam, to be used for a long time and to the foam.

### BACKGROUND ART

Previously, liquid-phase cosy etic compositioncompositions have been mainly filled in vacuum containers, pump containers, or glass containers to be distributed and stored. However, there is a disadvantage that such containers have poor portability. Recently, as the need to easily put on or touch up make-up even outdoors increases. demand for portable liquid-phase cosmetic compositions is growing.

As a container for easily carrying liquid-phase cosmetic compositions, a pact-type container may be contemplated. To allow a liquid-phase cosmetic composition to be contained in a pact-type container, it is required to consider whether a carrier for the cosmetic composition is applicable to the pact-type container, whether the cosmetic composition is packed sufficiently into the carrier, whether the carrier impregnates the cosmetic composition homogeneously for a long time, and whether an adequate amount of the cosmetic composition is discharged from the carrier as desired, or the like.

To meet these considerations, foams were prepared by foaming synthetic resins, and the foams were found to have excellent packing ability, impregnating ability, and dischargeability. Therefore, these synthetic resin foams, for example, ester-based foams and ether-based foams were used as carriers for liquid-phase cosmetic compositions.

However, with regard to these previous foams used as carriers for liquid-phase cosmetic compositions, the ether-based foams are resistant to water but weak to oil, and therefore, corrosion occurs to generate a problem of weak oil resistance, and the ester-based foams are resistant to oil but weak to water, and therefore, corrosion occurs to generate a problem of weak water resistance. For these reasons, the foams are easy to crumble when used as carriers for a long time, leading to a problem that the foams lose their functions to be unusable.

### DETAILED DESCRIPTION OF THE INVENTION

### Technical Problem

An object of the present invention provides a method of preparing a foam capable of impregnating ar liquid-phase cosmetic material, in which the method increases hydrolysis resistance of a polyester sponge and ensures a balance between water resistance and oil resistance at the same time, and the foam.

Further, another object of the present invention provides a method capable of producing a foam having a uniform pore size, and excellent fluffiness, softness, flexibility and elasticity, and excellent packing ability to pack the liquid-phase cosmetic material, excellent impregnating ability to impregnate the liquid-phase cosmetic material homogeneously for a long time, and high dischargeability to discharge an adequate amount of the liquid-phase cosmetic composition when the liquid-phase cosmetic material is taken, and excellent durability even after impregnating the iquid-phase cosmetic material.

### Technical Solution

To achieve the above objects, a method of preparing a foam fear impregnating a liquid-phase cosmeticmaterial of the present invention is characterized by including:
(a) preparing a polyester polyol by putting the polyester polyol in a chamber, followed by deflation at a temperature of 55˜65° C. and mitigation under conditions of a pressure of 0.1˜0.2 kPa for 60 minutes, and then lowering the temperature to 25˜35°, followed by supply of nitrogen and, mitigation under conditions of a pressure of 2˜3 kPa for 48 hours;
(b) preparing a polyether polyol by putting the polyether polyol in a chamber, followed by mitigation under conditions of a temperature of 15˜25° C. and a pressure of 0.1˜0.2 kPa for 60 minutes, supply of nitrogen, and mitigation under conditions of a pressure of 2˜3 kPa for 48 hours;
(c) injecting and preparing a foaming agent, a catalyst,and surfactant in tanks according to capacity demands, respectively;
(d) sequentially putting, in a mixing and stirring tank, the polyester polyol, polyether polyol, foaming agent, catalyst, and surfactant according to a mixing ratio, and then performing continuous foaming with stirring at 5000 rpm under conditions of a temperature of 22˜24° C. and a tank internal pressure of 3 kPa;
(e) aging, for 48 hours a foam formed after the completion of the foaming step; and
(f) finishing the foam to manufacture a product.

Herein, the mixing of step (d) is characterized in that 0.2˜0.4 parts by weight of the foaming agent, 0.2˜0.4 parts by weight of the catalyst, and 2˜4 parts by weight of the surfactant are mixed, based on 100 parts by weight of a mixture which is obtained by mixing the polyester polyol and the polyether polyol at a weight ratio of 3:7˜4:6.

Herein, the foaming agent is characterized in that it is any one selected from the group consisting of HCFC-141b, HCFC-42b, and HCF-22 available from Soo Kyung Chemical Co., Ltd.

Herein, the catalyst is characterized in that it s any one selected from the group consisting of TEDA, DETDA, 33lv, A-1, A-99, and TEOA available from Soo Kyung Chemical Co., Ltd.

Herein, the surfactant is characterized in that it is any one selected from the group consisting of AK8818, AK8803 and AK8806 available from Soo Kyung Chemical Co., Ltd.

Further, the present invention provides a foam for impregnating a liquid-phase cosmetic material, characterized in that the foam is prepared by the above-described preparation method and a mixing ratio of the polyester polyol and the polyether polyol is any one of 3:7 and 4:6.

The present invention provides a foam for impregnating a liquid-phase cosmetic material, the foam having physical properties of an average pore size of 900˜1,100 μm, a pore number of 60˜80 ppi (pore per inch), a density of 40˜45 kg/m³, a repulsive elasticity of 40˜45%, a compressive strain of 30˜40%, a hardness of 75˜80°, a tensile strength of 220˜230 Kpa, and an absorption rate of 300˜400%.

### ADVANTAGEOUS EFFECTS OF THE INVENTION

A preparation method provided by the present invention is advantageous in terms of providing a foam for impregnating a liquid-phase cosmetic material, the foam having a uniform pore size, and excellent fluffiness, softness, flexibility and elasticity.

Further, the foam prepared by the preparation method of the present invention has advantages of having excellent packing ability to pack the liquid-phase cosmetic material, excellent impregnating ability to impregnate the liquid-phase cosmetic material homogeneously for a long time, and high dischargeability to discharge an adequate amount of the liquid-phase cosmetic composition when the liquid-phase cosmetic material is taken, and of maintaining excellent durability even after impregnating the liquid-phase cosmetic material.

### BEST MODE

Hereinafter, the present invention will be described more detail with reference to the accompanying drawings.

The present invention provides a method of preparing a foam or impregnating a liquid-phase cosmetic material, which is used to impregnate a liquid-phase or solid-phase cosmetic composition such as a solution, an emulsion, a gel, a cream, a suspension, etc., and the method of preparing a foam for impregnating a liquid-phase cosmetic material according to the present invention is characterized by including the following steps:
(a) preparing a polyester polyol by putting the polyester polyol in a chamber, followed by deflation at a temperature of 55˜65° C. and mitigation under conditions of a pressure of 0.1˜0.2 kPa for 60 minutes, and then lowering the temperature to 25˜35°, followed by supply of nitrogen and mitigation under conditions of a pressure of 2˜3 kPa for 48 hours;
(b) preparing a polyether polyol by putting the polyether polyol in a chamber, followed by mitigation under conditions of a temperature of 15˜25° C. and a pressure of 0.1˜0.2 kPa for 60 minutes, supply of nitrogen, and mitigation under conditions of a pressure of 2˜3 kPa for 48 hours;
(c) injecting and preparing a foaming agent, a catalyst, and a surfactant in tanks according to capacity demands, respectively;
(d) sequentially putting, in a mixing and stirring tank, the polyester polyol, polyether polyol, foaming agent, catalyst, and surfactant according to a mixing ratio, and then stirring a mixture at 5000 rpm under conditions of a temperature of 22˜24° C. and a tank internal pressure of 3 kPa, followed by putting the mixture in a mold and performing continuous foaming;
(e) aging, for 48 hours, a foam formed after the completion of the foaming step; and
(f) finishing the foam to manufacture a product.

According to the present invention, the mixing of step (d) is characterized in that 0.2˜0.4 parts by weight of the foaming agent, 0.2˜0.4 parts by weight of the catalyst, and 2˜4 parts by weight, of the surfactant are mixed, based on 100 parts by weight of a mixture which is obtained by mixing the polyester polyol and the polyether polyol at a weight ratio of 3:7˜4:6.

In this regard, when the mixing ratio of the polyester polyol and the polyether polyol exceeds a critical value, there are disadvantages that a pore size of the finally produced foam is not homogeneous, and a sufficient density is not obtained to deteriorate an absorption rate.

Further, there are problems that because of low repulsive elasticity, sense of use is decreased after impregnating the liquid-phase cosmetic material, and the liquid-phase cosmetic material is not uniformly discharged after being impregnated. The pore size is one of important factors in impregnating the liquid-phase cosmetic material. Preferably, when the pores impregnating the liquid-phase cosmetic material are processed to have an average pore size of 900˜1,100 μm and also to satisfy a pore number of 60˜80 ppi, the foam have properties such as excellent abilities to impregnate, pack, and discharge the liquid-phase cosmetic material.

Further, it is preferred that the foam satisfies a repulsive elasticity of 40˜45% which allows the foam to be readily restored to its initial state after compression.

Further, the foam for impregnating the liquid-phase cosmetic material must have an appropriate compressive strain, and it is favorable that the foam satisfies the compressive strain of 30˜40%, which allows the foam to have soft sense of touch.

Meanwhile, to impregnate the liquid-phase cosmetic material, the foam must have appropriate density and hardness, tensile strength, and absorption rate, and preferably, the foam must have physical properties of the density of 40˜45 kg/m³, hardness of 75˜80°, tensile strength of 220˜230 Kpa, and absorption rate of 300˜400%, which allows the foam to impregnate a larger amount of cosmetic material.

According to the present invention, any foaming agent may be used, as long as it is commonly used in foaming synthetic resins. More preferably, to allow a final product, foam to have excellent physical properties for impregnating the liquid-phase cosmetic material, any one selected from the group consisting of HCFC-141b, HCFC-142b, and HCF-22 is favorably used to uniformly disperse and foam the raw materials without tangling.

According to the present invention, the catalyst functions to form homogeneous pores in foams by generating bubbles, and any one selected from the group consisting of TEDA, DETDA, 33lv, A-1, A-99, and TEOA is used, and TEDA is the most, preferably used. Further, the surfactant is added to prevent generation of large bubbles and poor bubbles, and any one selected from the group consisting of AK8818, AK8803, and AK8806 is favorably used. More favorably, AK8818 is preferably used.

According to the prepare ion method of the present invention as described above, provided is a foam for impregnating a liquid-phase cosmetic material, the foaming having physical properties of an average pore size of 900˜1,100 μm, a pore number of 60˜80 ppi (pore per inch), a density of 40˜45 kg/m³, a repulsive elasticity of 40˜45%, a compressive strain of 30˜40%, a hardness of 75˜80°, a tensile strength of 220˜230 Kpa, and an absorption rate of 300˜400%.

### MODE OF THE INVENTION

Hereinafter, the present invention will be described in more detail with reference to the preferred Examples. However, the following Examples are for illustrative purposes only, and the present invention is not intended to be limited by the following Examples, and various modifications may be made without departing from the scope of the claims of the present invention.

### Examples 1 and 2

1) Preparation of polyester polyol: a polyester polyol was prepared by putting the polyester polyol in a chamber, followed by deflation at a temperature of 55˜65° C. and mitigation under conditions of a pressure of 0.1˜0.2 kPa for 60 minutes, and then by lowering the temperature to 25˜35°, followed by supply of nitrogen and mitigation under conditions of a pressure of 2˜3 kPa for 48 hours.

2) Preparation of polyether polyol: a polyether polyol was prepared by putting the polyether polyol in a chamber, followed by mitigation under conditions of a temperature of 15˜25° C. and a pressure of 0.1˜0.2 kPa for 60 minutes, supply of nitrogen, and mitigation under conditions of a pressure of 2˜3 kPa for 48 hours.

3) Preparation of other additives: a foaming agent, a catalyst, and a surfactant were injected and prepared in tanks according to capacity demands, respectively.

4) Foaming and molding: the prepared polyester polyol, polyether polyol, foaming agent, catalyst, and surfactant were sequentially put in a mixing and stirring tank according to appropriate mixing conditions, and then continuous foaming was performed with stirring at 5000 rpm under conditions of a temperature of 22˜24° C. and a tank internal pressure of 3 kPa, followed by aging a foam thus formed for 48 hours and finishing the foam to have a size suitable for impregnating the liquid-phase cosmetic material.

In this regard, the polyester polyol and the polyether polyol were injected to satisfy a weight ratio of 3:7 (Example 1) and 4:6 (Example 2), and the foaming agent, the catalyst, and the surfactant were injected and mixed in an amount of 0.3 parts by weight, 0.3 parts by weight, and 3 parts by weight, respectively, based on 100 parts by weight of a mixture of the polyester polyol and the polyether polyol, followed by carrying out the foaming process. D19 was used as the foaming agent. A230 was used as the catalyst, and SL9205 was used as the surfactant.

### Comparative Examples 1 and 2

A known foam (Comparative Example 1) obtained by using only polyester polyol and a known foam (Comparative Example 2) obtained by using only polyether polyol were prepared.

Physical properties of the foams prepared by the above-disclosed Examples and Comparative Examples were compared and the results are given in the following Table 1.

TABLE 1

| | Experimental | Example | Example | Comparative | Comparative |
|---|---|---|---|---|---|
| Items | method | 1 | 2 | Example 1 | Example 2 |
| Density (kg/m³) | ISO 845 | 41.2 | 40.5 | 21.3 | 13.1 |
| Hardness (°) | JSI K6301A | 79 | 78 | 73 | 72 |
| Tensile strength | ISO 1798 | 222.32 | 235.6 | 250.8 | 210.4 |
| (Kpa) | | | | | |
| Repulsive elasticity (%) | JIS K6400 | 44 | 42 | 28 | 42 |
| Compressive strain (%) | JIS K6400 | 35 | 37 | 32 | 26 |
| Pore size (μm) | WI-QA-14 | 1,100 | 900 | 500 | 400 |
| Pore number (ppi) | WI-QA-14 | 80 | 60 | 55 | 60 |
| Absorption rate (%) | | 400 | 300 | 200 | 100 |
| Hydrolysis | ASTM D-6860 | Very | Very | Good | Poor |
| resistance | | good | good | | |
| Oil resistance | ASTM D-6860 | Very | Very | Poor | Good |
| | | good | good | | |

In Table 1, the pore size and pore number were measured according to WI-QA-14 (ASTM standard), and the absorption rate was measured by preparing samples of the prepared foams in a size of 3 cm×3 cm, drying the samples in an oven at 70° C. for 24 hours, and then measuring their initial weights (A), impregnating and storing the samples in distilled water at 25° C. for 48 hours, and then taking the samples out and wiping off water of the surface with a dust-free tissue, and then measuring their weights (B). Finally, the absorption rate was calculated by the following Equation 1:

Absorption rate (%)=(B−A)/A×100 (Equation 1)

Further, hydrolysis resistance was measured by a method known as a jungle test (ASTM D-6860), and the results are as given in the following Table 2. Oil resistance was measured by the jungle test (ASTM D-6880), and the results are as given in the following Table 3.

<Experimental Method>

T: 60° C.

R/H: 98%

Exposure-time (days): 15 days

TABLE 2

| | | | | | Temper- | | |
|---|---|---|---|---|---|---|---|
| | | | | | ature | Water | |
| Time | Ether | Ester | 3:7 | 4:6 | (° C.) | resistance | Note |
| 1 day | x | x | x | x | 60 | ∘ | No change |
| 10 days | x | x | x | x | 60 | ∘ | No change |
| 15 days | x | Δ | x | x | 60 | Δ | Ester started to |
| | | | | | | | change |
| 30 days | x | ∘ | x | x | 60 | x | Not for |
| | | | | | | | cosmetic use |

x: no deformation,
Δ: deformation started,
∘: deformation completed

TABLE 3

| | | | | | Temper- | | |
|---|---|---|---|---|---|---|---|
| | | | | | ature | Oil | |
| Time | Ether | Ester | 3:7 | 4:6 | (° C.) | resistance | Note |
| 1 day | x | x | x | x | 60 | ∘ | No change |
| 10 days | Δ | x | x | x | 60 | x | No change |
| 15 days | ∘ | x | x | x | 60 | x | Known foams |
| 30 days | ∘ | x | Δ | Δ | 60 | x | could not be |
| | | | | | | | used for |
| | | | | | | | cosmetics, but |
| | | | | | | | foams of the |
| | | | | | | | present |
| | | | | | | | invention |
| | | | | | | | could be used |
| | | | | | | | for cosmetics |

x: no deformation,
Δ: deformation started,
∘: deformation completed

According to the results of hydrolysis resistance and oil resistance shown in Tables 1 to 3, it can be seen that the foams of the present invention showed improved water resistance and oil resistance, whereas the known ester-based foam or ether-based foam composed of a single component was resistant to water but weak to oil, or resistant to oil but weak to water, respectively and therefore, they could not be used for a long time to generate a problem of a short life-time.

According to the results of Table 1, it can be seen that the method of preparing a foam for impregnating a liquid-phase cosmetic material provided by the present invention may be used to provide a foam having very excellent physical properties. That is, it can be seen that the foam provided according to the present invention has a uniform pore size, and excellent fluffiness, softness, flexibility and elasticity, and excellent packing ability to pack the liquid-phase cosmetic material, excellent impregnating ability to impregnate the liquid-phase cosmetic material homogeneously for a long time, and high dischargeability to discharge an adequate amount of the liquid-phase cosmetic composition when the liquid-phase cosmetic material is taken, and excellent durability even after impregnating the cosmetic material.

In particular, there are advantages that the foam has a high absorption rate to impregnate a large amount of cosmetic materials, and also, the foam easily impregnates the cosmetic materials.

1. A method of preparing a foam for impregnating a liquid-phase cosmetic material, the method comprising:
(a) preparing a polyester polyol by putting the polyester polyol in a chamber, followed by deflations at a temperature of 55˜65° C. and mitigation under conditions of a pressure of 0.1˜0.2 kPa for 60 minutes, and then lowering the temperature to 25˜25°, followed by supply of nitrogen and mitigation under conditions of a pressure of 2˜3 kPa for 48 hours;
(b) preparing a polyether polyol by putting the polyether polyol in a chamber, followed by mitigation under conditions of a temperature of 15˜25° C. and a pressure of 0.1˜0.2 kPa for 60 minutes, supply of nitrogen, and mitigation under conditions of a pressure of 2˜3 kPa for 48 hours;
(c) injecting and preparing a foaming agent, a catalyst, and a surfactant in tanks according to capacity demands, respectively;
(d) sequentially putting, in a mixing and stirring tank, the polyester polyol, polyether polyol, foaming agent, catalyst, and surfactant according to a mixing ratio, and then performing continuous foaming with stirring at 5000 rpm under conditions of a temperature of 22˜24° C. and a tank internal pressure of 3 kPa;
(e) aging, for 48 hours, a foam formed after the completion of the foaming step (d); and
(f) finishing the foam to manufacture product.

2. The method of claim 1, wherein in the mixing of step (d), 0.2˜0.4 parts by weight of the foaming agent, 0.2˜0.4 parts by weight of the catalyst, and 2˜4 parts by weight of the surfactant are mixed, based on 100 parts by weight, of a mixture which is obtained by mixing the polyester polyol and the polyether polyol at a weight ratio of 3:7˜4:6.

3. The method of claim 1, wherein the foaming agent is any one selected from the group consisting of HCFC-141b, HCFC-142b, and HCF-22.

4. The method of claim 1, wherein the catalyst is any one selected from the roup consisting of TEDA, DETDA, 33lv, A-1, A-99, and TEOA.

5. The method of claim 1, wherein the surfactant is any one selected from the group consisting of AK8818, AK8803, and AK8806.

6. A foam for impregnating a liquid-phase cosmetic material, wherein a mixing ratio of polyester polyol and polyether polyol is any one of 3:7 and 4:6.

7. The foam for impregnating a liquid-phase cosmetic material of claim 6, wherein the foam has an average pore size of 900˜1100 μm, a pore number of 60˜80 ppi (pore per inch), a density of 40˜45 kg/m³, a repulsive elasticity of 40˜45%, a compressive strain of 30˜40%, a hardness of 75˜80°, a tensile strength of 220˜230 Kpa, and an absorption rate of 300˜400%.
